(19) United States
(12) Patent Application Publication
(10) Pub. No.: US 2023/0101963 A1
(43) Pub. Date: Mar. 30, 2023

(54) TREATMENT OF MEDICAL INDICATION

(71) Applicant: VALLAURIX TRADING SARL MC, MONACO (MC)

(72) Inventor: Philippe WOLGEN, MONACO (MC)

(21) Appl. No.: 17/800,994

(22) PCT Filed: Feb. 23, 2021
(86) PCT No.: PCT/EP2021/054468

§ 371 Date: Aug. 19, 2022

(30) Foreign Application Priority Data
Feb. 23, 2020 (EP) 20158912.4
Aug. 31, 2020 (EP) 20193759.6
Oct. 26, 2020 (EP) 20203949.1

(51) Int. Cl. A61K 38/34; A61P 9/10

(52) U.S. Cl. CPC A61K 38/34; A61P 9/10

(57) ABSTRACT

The present invention is directed to hMC1R and hMC4R agonist compounds such as afamelanotide, bremelanotide and pharmaceutically acceptable salts thereof for use in treatment of a human subject suffering from cerebrovascular accident (CVA), in particular AIS.

### TECHNICAL FIELD

The present invention relates to alpha-MSH analogues for use in the treatment of a human subject suffering from a cerebrovascular accident (CVA), particularly arterial ischemic stroke (AIS).

### BACKGROUND TO THE INVENTION

There is a high medical need for improvement of treatment outcome of CVA and particularly AIS in human subjects. While animal model may be used to evaluate candidates for treatment, the large number of failures of pharmaceutical agents in subsequent clinical development of for instance CVA (and specifically AIS) proves that these animal models have limited value in predicting effective medication in the treatment of human subjects. This represents a major complication for development of new medication for treatment of CVA and in particular AIS.

Accordingly, there is a high unmet medical need to improve the treatment of CVA and particularly AIS in humans with better clinical outcomes.

### SUMMARY OF THE INVENTION

The present invention is also directed to a compound (“the compound of the invention”) for use in the treatment of a human subject suffering from cerebrovascular accident (CVA) and in particular AIS wherein the compound is a selective, human MelanoCortin-1-Receptor (hMC-1-R) and human MelanoCortin-4-Receptor (hMC4R) agonist (a selective hMC1R and hMC4R agonist).

Preferably, the hMC1R and hMC4R agonist of the invention is Blood-Brain-Barrier (BBB) permeable.

Preferably, the compound of the invention is administered in the form of an injectable preparation or dosage form. Preferably, the compound of the invention is administered in an immediate or controlled release injection. Preferably, the compound of the invention is injected subcutaneously. Preferably, the compound of the invention is administered with a dose of from 0.0007 to 1.5 mg/kg/day for at least 1 day, wherein kg represents the weight of the human subject in kilograms.

Preferably, the compound of the invention is administered at a dose of from 0.05 to 100 mg. In one preferred embodiment, the invention relates to ischemic CVA (alternatively called AIS). In another preferred embodiment, the invention relates to hemorrhagic CVA. Preferably, the human subject is treated with a tissue plasminogen activator (tPA) and subsequently or prior treated with the compound of the invention.

In a further embodiment, the invention is directed to a composition comprising the compound of the invention for use in treatment of a human subject suffering from cerebrovascular accident (CVA), in particular AIS.

In a further embodiment, the invention is directed to a method of treatment of a human subject suffering from cerebrovascular accident (CVA), in particular AIS wherein the human subject is treated with the compound of the invention.

In a further embodiment, the invention is directed to the use of the compound of the invention for the manufacturing of a medicament for the treatment of a human subject suffering from cerebrovascular accident (CVA), in particular AIS.

Preferably, the compound of the invention is selected from afamelanotide, a pharmaceutically acceptable salt of afamelanotide, bremelanotide, a pharmaceutically acceptable salt of bremelanotide and mixtures thereof.

Preferably, the present invention is directed to a compound for use in the treatment of a human subject suffering from cerebrovascular accident (CVA), in particular AIS, wherein the compound is afamelanotide or a pharmaceutically acceptable salt thereof.

Preferably, afamelanotide or a pharmaceutically acceptable salt thereof is administered in the form of an injectable preparation or dosage form. Preferably, afamelanotide or a pharmaceutically acceptable salt thereof is administered in an immediate or controlled release injection. Preferably, afamelanotide or a pharmaceutically acceptable salt thereof is injected subcutaneously. Preferably, afamelanotide or a pharmaceutically acceptable salt thereof is administered with a dose of from 0.03 to 1.5 mg/kg/day for at least 1 day, wherein kg represents the weight of the human subject in kilograms. Preferably, afamelanotide or a pharmaceutically acceptable salt thereof is administered at a dose of from 2.5 to 100 mg. In one preferred embodiment, the invention relates to ischemic CVA (generally called AIS). In another preferred embodiment, the invention relates to hemorrhagic CVA. Preferably, the human subject is treated with a tissue plasminogen activator (tPA) and subsequently or prior treated with afamelanotide or a pharmaceutically acceptable salt thereof. Preferably, afamelanotide is administered as a subcutaneously administered 16 mg implant on day 0 and on day 1, more preferably also on day 7 and on day 8.

In a further embodiment, the invention is directed to a composition comprising afamelanotide or a pharmaceutically acceptable salt thereof for use in treatment of a human subject suffering from cerebrovascular accident (CVA), in particular AIS.

In a further embodiment, the invention is directed to a method of treatment of a human subject suffering from cerebrovascular accident (CVA), in particular AIS, wherein the human subject is treated with afamelanotide or a pharmaceutically acceptable salt thereof.

In a further embodiment, the invention is directed to the use of afamelanotide or a pharmaceutically acceptable salt thereof for the manufacturing of a medicament for the treatment of a human subject suffering from cerebrovascular accident (CVA), in particular AIS.

Preferably, the present invention is directed to a compound for use in the treatment of a human subject suffering from cerebrovascular accident (CVA), in particular AIS wherein the compound is bremelanotide or a pharmaceutically acceptable salt thereof.

Preferably, bremelanotide or a pharmaceutically acceptable salt thereof is administered in the form of an injectable preparation or dosage form. Preferably, bremelanotide or a pharmaceutically acceptable salt thereof is administered in an immediate or controlled release injection. Preferably, bremelanotide or a pharmaceutically acceptable salt thereof is injected subcutaneously. Preferably, bremelanotide or a pharmaceutically acceptable salt thereof is administered with a dose of from 0.0007 to 0.04 mg/kg/day for at least 1 day, wherein kg represents the weight of the human subject in kilograms.

Preferably, bremelanotide or a pharmaceutically acceptable salt thereof is administered at a dose of from 0.05 to 2.5 mg. In one preferred embodiment, the invention relates to ischemic CVA (AIS). In another preferred embodiment, the invention relates to hemorrhagic CVA. Preferably, the human subject is treated with a tissue plasminogen activator (tPA) and subsequently or prior treated with bremelanotide or a pharmaceutically acceptable salt thereof.

In a further embodiment, the invention is directed to a composition comprising bremelanotide or a pharmaceutically acceptable salt thereof for use in treatment of a human subject suffering from cerebrovascular accident (CVA), in particular AIS.

In a further embodiment, the invention is directed to a method of treatment of a human subject suffering from cerebrovascular accident (CVA), in particular AIS wherein the human subject is treated with bremelanotide or a pharmaceutically acceptable salt thereof.

In a further embodiment, the invention is directed to the use of bremelanotide or a pharmaceutically acceptable salt thereof for the manufacturing of a medicament for the treatment of a human subject suffering from cerebrovascular accident (CVA), in particular AIS.

Without wishing to be bound by any theory, it is believed that afamelanotide or bremelanotide (or a pharmaceutically acceptable salt and/or mixtures thereof) benefits human subjects in terms of treatment symptoms and outcome for CVA, in particular AIS including higher survival rate and/or improved motor functioning and/or improved cognitive functioning and/or decreased ischemic, necrotic zones, in the short and/or longer term. Surprising, the inventor realized from the side-effect profile that the compounds of the invention (afamelanotide, bremelanotide and their respective pharmaceutically acceptable salts) show central activity in human, indicating the compound passes the human blood-brain-barrier. Importantly, the inventor then realized that these compounds have selective agonist affinity for the hMC1R and hMC4R (vs low activity on hMC3R and hMC5R and none on hMC2R) and, after crossing the BBB, can beneficially associate with these specific receptors in the brain (specifically in nuclei of the hypothalamus), triggering the Hypothalamus-Pituitary-Adrenal (HPA) axis and leading to effects on the blood vessels, a vaso-active response influencing the vascular endothelium. Thus, interestingly, the inventor surprisingly realized that these specific hMC1R and hMC4R agonist compounds have central activity leading to benefits for patients suffering from blood vessel related diseases, in particular CVA and especially patients suffering from ischemic CVA (also called AIS).

### DETAILED DESCRIPTION OF THE INVENTION

According to the invention, we have surprisingly found that afamelanotide, a pharmaceutically acceptable salt of afamelanotide, bremelanotide, a pharmaceutically acceptable salt of bremelanotide and/or mixtures thereof is effective in treatment of human subjects suffering from cerebrovascular accident (CVA), in particular AIS.

For the purpose of this invention, the following abbreviations are used: Ala = alanine, Arg = arginine, Dab - 2,4-diaminobutyric acid, Dpr = 2,3- diaminopropionic acid, Glu = glutamic acid, Gly = glycine, His = histidine, HomoArg - homoarginine (one more —CH₂— unit in the alkyl chain than Arg), norArg -norarginine (one fewer —CH2 unit in the alkyl chain than Arg), Lys = lysine, Met = methionine, Nle = norleucine, Orn = ornithine, Phe = phenylalanine, (pNO2)Phe = paranitrophenylalanine, Plg = phenylglycine, Pro = proline, Ser = serine, Trp = tryptophan, TrpFor = N¹ formyl-tryptophan, Tyr = tyrosine, Val = valine.

For the purpose of this invention, all peptides are written with the N-terminus on the left and the C-terminus on the right; the prefix “D” before an amino acid designates the D-isomer configuration, and unless specifically designated otherwise, all amino acids are in the L-isomer configuration. All peptide and peptide derivatives are written with the acylated amino terminal end at the left, the N-terminus and the amidated carboxyl terminal at the right, the C-terminus. As will be understood, the acylated amino terminal end may be replaced by another group according to the invention but the orientation of the peptides and peptide derivatives remains the same. Following common convention, the first amino acid on the left is located at position 1, for instance, Nle (1) indicating that Nle is positioned at the N terminal end (on the left). The skilled person will understand that reference in this document to peptide molecules includes reference to derivatives thereof such as peptides that have been synthesized or comprise synthetic amino acids.

For the purpose of the invention, the association constant of a compound with the human MC1R and MC4R reflects the affinity of that compound for the human MC1R or human MC4R receptor. This compound preferably has agonist affinity and this can be determined by standard methods described in the art.

For the purpose of the invention, the BBB permeability refers to the ability of a compound to cross the Blood-Brain-Barrier (BBB) that is intact, i.e. not leaking or otherwise compromised. Permeability can be determined based on presence of central effects (or side-effects). A compound with BBB permeability can show central effects or side-effects.

CVA (often called “stroke”) is an acute and life-threatening medical condition. Causes for CVA can be ischemia (restricted blood supply) and hemorrhage (bleeding) in the brain. Sometimes TIA (Transient Ischemic Attack) may be included as separate cause of CVA. Ischemic CVA (called Arterial Ischemic Stroke or “AIS”) represents the majority of CVAs and is caused by obstruction of critical blood supply, for instance a blood clot occluding a cerebral artery. A hemorrhagic CVA occurs when a blood vessel in the brain ruptures, for instance due to high blood pressure, vascular malformations or tumor tissue. CVA may also be caused by trauma. Generally, CVA results in lack of oxygen and nutrients in the brain tissue and/or in damaged brain tissue in the vascularized area beyond the obstruction or hemorrhage.

The present invention is directed at treatment of CVA, in particular AIS. The invention is further directed to treatment of CVA (in particular AIS) symptoms and/or causes; to reducing the complications of CVA, in particular AIS; and/or to reducing the occurrence of complications of CVA, in particular AIS.

The compound of the invention preferably has human MC-1-R and MC-4-R agonist activity. hMC1R and hMC4R agonist activity is defined as having primary agonist (stimulating) activity on the human MC1R and on the MC-4-R relative to each of the hMC-3-R, the hMC-5-R and/or the hMC-2-R. Preferably, the agonist activity of the compound on both hMC1R and hMC4R is high relative to its agonist activity hMC3R, hMC5R and hMC2R. For instance, in-vitro tests on the human MC1R and the MC4R showed that bremelanotide is more than 5.05x more potent than alpha-MSH on the human MC1R while afamelanotide showed more potency than bremelanotide. Also, bremelanotide was 94x more potent than alpha-MSH on the human MC4R while afamelanotide was 12.5x more potent compared to alpha-MSH.

Preferably, the hMC1R and hMC4R agonist of the invention is Blood-Brain-Barrier (BBB) permeable. This means that the compound of the invention passes through the intact human BBB, as such allowing for central activity after peripheral administration. BBB permeable is the ability of a compound to pass the BBB that is intact and for instance not leaking or otherwise compromised. Permeability can be determined based on presence of central effects (or side-effects) after peripheral administration of the compound.

Preferably, the compound of the invention is selected from afamelanotide, a pharmaceutically acceptable salt of afamelanotide, bremelanotide, a pharmaceutically acceptable salt of bremelanotide and mixtures thereof.

Afamelanotide is a synthetic analogue of alpha-MSH and is also known by other names such as NDP-MSH, Melanotan I, CUV1647 and can be represented by the formula [Nle⁴, D-Phe⁷]-alpha-MSH. This means that Nle replaces Met in the 4^{th} position and D-Phe replaces Phe in the 7^{th} position in natural alpha-MSH hormone.

Bremelanotide is a synthetic, cyclic heptapeptide analogue of alpha-MSH and can be represented by the formula structure C50H68N14O10 with a molecular weight of 1052.2. Bremelanotide is preferably used as acetate salt, preferably in the form represented by Ac-Nle-cyclo-(Asp-His-D-Phe-Arg-Trp-Lys-OH) ● xCH3COOH ((1≤ x ≤ 2).

For the purpose of this invention, afamelanotide and bremelanotide may be used as such or in the form of a pharmaceutically acceptable salt thereof. Preferred examples of such salts are acetate, trifluoroacetate, sulfate, and chloride salts. The acetate salt is generally most preferred.

Afamelanotide, bremelanotide or a pharmaceutically acceptable salt or mixtures thereof is preferably administered to the human subject suffering from cerebrovascular accident (CVA), in particular AIS, in a solution. Afamelanotide, bremelanotide or a pharmaceutically acceptable salt or mixtures thereof may for instance be administered as an infusion, an intra-muscular injection, an intra-peritoneal injection or a subcutaneous injection or an injection in the cerebrum (intracerebral injection). Preferably, afamelanotide, bremelanotide or a pharmaceutically acceptable salt or mixtures thereof is administered as an injection, more preferably as an immediate release injection. Preferably, afamelanotide, bremelanotide or a pharmaceutically acceptable salt or mixtures thereof is injected subcutaneously.

Preferably, afamelanotide, bremelanotide or a pharmaceutically acceptable salt or mixtures thereof is administered with a dose of from 0.007 to 1.5 mg/kg/day, wherein kg represents the weight of the human subject in kilograms. The preferred dose (preferably of afamelanotide) is preferably at least 0.1 mg/kg/day, more preferably at least 0.25 mg/kg/day, most preferably at least 4 mg/kg/day and preferably up to 1 mg/kg/day, more preferably up to 0.85 mg/kg/day, most preferably up to 0.7 mg/kg/day, for instance 0.6 mg/kg/day. Preferred doses for bremelanotide is at least 0.0007 mg/kg/day, more preferably at least 0.001 mg/kg/day, most preferably at least 0.005 mg/kg/day, particularly preferred is at least 0.001 mg/kg/day and preferably at most 0.1 mg/kg/day, more preferably at most 0.8 mg/kg/day and most preferably at most 0.04 mg/kg/day.

Preferably, afamelanotide, bremelanotide or a pharmaceutically acceptable salt or mixtures thereof is administered at a dose (preferably a daily dose) of from 0.05 to 100 mg. For instance, the dose can be at least 0.1 mg. Preferably, the dose (particularly of afamelanotide) is at least 5 mg, more preferably at least 10 mg, most preferably at least 20 mg, particularly preferred is at least 22 mg and for instance up to 100 mg, preferably up to 90 mg, more preferably up to 70 mg, most preferably up to 50 mg, particularly preferred is up to 35 mg. These doses also preferably represent the daily dose. Bremelanotide is preferably daily dosed at levels of at least 0.01 mg, more preferably at least 0.05 mg, most preferably at least 0.1 mg and preferably up to 5 mg, more preferably up to 3 mg, most preferably up to 2.5 mg.

Afamelanotide, bremelanotide or a pharmaceutically acceptable salt or mixtures thereof is administered preferably at least once a day and preferably up to three times a day, more preferably once a day.

Preferably, afamelanotide, bremelanotide or a pharmaceutically acceptable salt or mixtures thereof is administered to the human subject for a period of at least 2 days, more preferably at least 5 days, most preferably at least 8 days. Preferably, afamelanotide, bremelanotide or a pharmaceutically acceptable salt or mixtures thereof is administered for a period of up to 50 days, more preferably up to 20 days and most preferably up to 12 days, for instance 10 days.

Preferably, afamelanotide is administered as a 16 mg implant. Preferably, according to the present invention, afamelanotide bremelanotide, or a pharmaceutically acceptable salt or mixtures thereof is administered to the human subject on day 0, more preferably also day 1, most preferably also on day 7 and particularly preferred also on day 8. Alternatively, dosing according to the present invention preferably starts on day 1, and then more preferably also on day 2. As will be understood, day 0 is the patient is diagnosed with CVA (or specifically AIS).

Further preferred dosing schedules according to the present invention for afamelanotide, bremelanotide, or a pharmaceutically acceptable salt or mixtures thereof according to the present invention are as follows:
Dosing option 1: dosing days 0, 1, 7, and 8;
Dosing option 2: dosing days 0, 1, 3, 4, 7, and 8;
Dosing option 3: dosing days 1, 2, 5, 6, 7, and 8;
Dosing option 4: dosing days 1, 2, 6, 7, 8, and 9; or
Dosing option 5: dosing days 0, 1, 2, 7, 8, and 9.

Preferably, afamelanotide, bremelanotide or a pharmaceutically acceptable salt or mixtures thereof is used to treat a human subject suffering from ischemic CVA (also called AIS) or from CVA due to a hemorrhage.

Preferably, human subjects suffering from ischemic CVA (also called AIS) are treated with a tissue plasminogen activator (tPA) and subsequently or prior with afamelanotide, bremelanotide or a pharmaceutically acceptable salt or mixtures thereof. Preferably, tPA is administered within 4.5 hours of the CVA event (specifically the AIS event). Preferably, afamelanotide, bremelanotide or a pharmaceutically acceptable salt or mixtures thereof is administered thereafter. Preferably, afamelanotide, bremelanotide or a pharmaceutically acceptable salt or mixtures thereof is administered after 4.5 hours of the CVA event (specifically the AIS event) occurring.

Preferably, afamelanotide, bremelanotide or a pharmaceutically acceptable salt or mixtures thereof is administered as a composition. The composition is preferably liquid and preferably transparent. Afamelanotide and bremelanotide are both preferably included in the form of salt, preferably the acetate salt. Preferably, the composition comprises afamelanotide or bremalenotide in salt form. Preferably, the composition comprises one or more pharmaceutically acceptable ingredients. Examples of pharmaceutically acceptable ingredients are water, salt, polymer, fatty acid, sugar, and surfactant. Preferably, the pharmaceutically acceptable ingredients include water and salt.

A further embodiment of the present invention relates to a method of treatment of human subjects suffering from cerebrovascular accident (CVA), in particular AIS, wherein the human subject is treated with afamelanotide or a pharmaceutically acceptable salt thereof, bremelanotide or a pharmaceutically acceptable salt thereof or mixtures thereof.

A further embodiment of the present invention relates to the use of afamelanotide, bremelanotide or a pharmaceutically acceptable salt or mixtures thereof for the manufacturing of a medicament for the treatment of a human suffering from cerebrovascular accident (CVA), in particular AIS.

### Examples

Compositions comprising afamelanotide acetate were prepared and administered by injection on a daily basis to subjects diagnosed with cerebrovascular accident (CVA), in particular AIS at dosages indicated in the above description for periods of up to 10 or up to 20 days. Meanwhile neuroimaging such as CT-scanning, perfusion-CT, MRI and EEG techniques were used to monitor disease state, pathology, progression and recovery of the subjects, allowing for instance determination of ischemic, necrotic zones and cerebral functions. Motor functions are also subject of monitoring.

Specifically, acute AIS (Arterial Ischemic Stroke) human subjects with distal [M2 and beyond] arterial LVO or perforator occlusion) were diagnosed on day 0. At the screening and admission visit (Day 0) patients were evaluated through computed tomography perfusion (CTP) of the cerebrum, allowing comparisons to Magnetic Resonance Imaging (MRI) [Diffusion Weighted Imaging (DWI) and Fluid Attenuated Inversion Recovery (FLAIR)] on subsequent days. Neurological functions were assessed on using the National Institutes of Health Stroke Scale (NIHSS). Further, use of the modified Rankin Scale (mRS) and the Montreal Cognitive Assessment (MoCA) allowed making comparisons.

Subjects and were treated with a subcutaneously administer 16 mg afamelanotide implant on day 0, on day 1, on day 7 and on day 8.

During and after treatment, adverse events were recorded. Further, positive results were obtained from evaluation of changes in reperfusion of the ischemic core and penumbral ischemic zone (salvageable tissue), for instance by hemodynamics analysis and also from analysis of neurological functions, cognitive functions and daily activities.

A 72 year old male was admitted to hospital after 5 hours following sudden loss of consciousness discovered by the daughter who had found her father on the floor of the living room. Upon admission, his pulse was 98 BPM, tension 120/50 and he hardly responded to external stimuli.

Upon diagnosis, computed tomography perfusion scan (CTP) showed a thrombus in the middle cerebral artery (MCA). The dislodged thrombus (clot) was located in an opercular branch of the MCA at M3 level. The patient had suffered an ischemic stroke (AIS) at higher regions of the brain causing hemiparesis, loss of speech and neurological deficit.

The patient was ineligible for recombinant Tissue Plasminogen Activator (r-TPA) due to the time lapsed between infarction and admission. Although tenecteplase as intravenous would have been the first choice of therapy in combination with endovascular thrombectomy (EVT), the location of the clot distal of the M2 branch of the MCA was an absolute contra-indication.

It was decided to administer the first subcutaneous afamelanotide 16 mg implant formulation on day of admission (day 0) followed by a second implant dose 24 hours later (day 1).

On day 4, an MRI scan by means of diffusion weighted imaging (DWI) and fluid inversion attenuated inversion recovery was performed to visualize the core infarct (necrotic tissue) and penumbra (surrounding brain tissue at risk). The MRI scan was compared with the CTP performed on day 0.

The radiological findings learned that there was an ischemic lesion visible by DWI without parenchymal hyperintensity on FLAR sequence. In reconstructing the images and quantification of blood flow and volume it was found that improvement of the r-cerebral blood flow (r-CBF) had improved from 20% to 25% in three days following two doses of afamelanotide. The Tmax had improved from 11 seconds to 8 seconds.

On day 7, a third afamelanotide implant was administered followed by a fourth on day 8. On day 9, a new MRI was performed to ascertain the changes in hypoxic brain tissue, with special interest for the ischemic core and penumbra.

These parameters indicated a treatment effect from afamelanotide, reaffirmed by the improvement of neurological deficit observed on day 4 and day 9 following the stroke (AIS).

1. A method of treatment of a human subject suffering from cerebrovascular accident (CVA), comprising:
administering an effective amount of a compound to the human subject, wherein the compound is a selective human MelanoCortin-1-Receptor and human MelanoCortin-4-Receptor (hMC4R) agonist.

2. The method according to claim 1, wherein the hMClR and hMC4R agonist is Blood-Brain-Barrier (BBB) permeable.

3. The method according to claim 1, wherein the compound is selected from the group consisting of afamelanotide, a pharmaceutically acceptable salt of afamelanotide, bremelanotide, a pharmaceutically acceptable salt of bremelanotide and mixtures thereof.

4. The method according to claim 1, wherein the compound is afamelanotide or a pharmaceutically acceptable salt thereof.

5. The method according to claim 1, wherein the compound is bremelanotide or a pharmaceutically acceptable salt thereof.

6. The method according to claim 1, wherein the compound is administered in the form of an injectable.

7. The method according to claim 1, wherein the compound is administered in an immediate or controlled release injection.

8. The method according to claim 1, wherein the compound is injected subcutaneously.

9. The method according to claim 1, wherein the compound is administered with a dose of from 0.0007 to 1.5 mg/kg/day for at least 1 day, wherein the kg represents the weight of the human subject in kilograms.

10. The method according to claim 1, wherein the compound is administered at a dose of from 0.05 to 100 mg.

11. The method according to claim 1, wherein the subject suffers from ischemic CVA (AIS) and/or hemorrhagic CVA.

12. The method according to claim 1, wherein the method comprises administering a tissue plasminogen activator (tPA) and subsequently or prior administering the compound, wherein the compound is selected from the group consisting of afamelanotide, a pharmaceutically acceptable salt of afamelanotide, bremelanotide, a pharmaceutically acceptable salt of bremelanotide and mixtures thereof.

13. The method according to claim 3, wherein the afamelanotide is administered as a subcutaneously administered 16 mg implant on day 0 and on day 1.

14. (canceled)

15. (canceled)
